(19) United States
(12) Patent Application Publication
(10) Pub. No.: US 2024/0023799 A1
(43) Pub. Date: Jan. 25, 2024

(54) Dental Cheek And Tongue Retracting Device And Method

(71) Applicant: Joseph Klug, Waianae, HI (US)

(72) Inventor: Joseph Klug, Waianae, HI (US)

(21) Appl. No.: 17/867,836

(22) Filed: Jul. 19, 2022

(51) Int. Cl. A61B 1/24

(52) U.S. Cl. CPC A61B 1/24

(57) ABSTRACT

A dental cheek and tongue retracting device and method for retaining in a fixed position within a mouth of a user includes a pad being used in a dental procedure. The pad is made of a cotton material and the pad is soft within a mouth of a user wherein providing comfort. A triangular shape of the pad includes a plurality of sides thus each of the sides is a flat surface. The flat surface of each of the sides assists in retaining the pad within a fixed position within the mouth of the first user. Furthermore, a plurality of points of the pad is a junction between the plurality of sides of the triangular shape of the pad.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to dental retractor device and more particularly pertains to a new dental retractor device for retaining in a fixed position within a mouth of a user.

### (2) Description of Related Art Including Information Disclosed under 37 CFR 1.97 and 1.98

The prior art relates to dental retractor devices. The prior art includes a variety of dental retractor devices being an absorbent material. Furthermore, the prior art relates to a variety of dental retractor devices being configured for retracting a tongue and cheek from a plurality of teeth of the user. Known prior art lacks a dental retractor device having a triangular shape being configured for retaining in a fixed position within the mouth of the user.

### BRIEF SUMMARY OF THE INVENTION

An embodiment of the disclosure meets the needs presented above by generally comprising a pad being configured for being used in a dental procedure. The pad is made of a cotton material and the pad is configured for being soft within a mouth of a user wherein providing comfort. A triangular shape of the pad includes a plurality of sides wherein each of the sides is configured for being a flat surface. The flat surface of each of the sides is configured for ameliorating in retaining the pad within a fixed position within the mouth of the first user. Furthermore, a plurality of points of the pad is a junction between the plurality of sides of the triangular shape of the pad.

Another embodiment of the disclosure includes a method comprising the step of inserting a pad within the mouth of the first user prior to nesting the pad from a tongue of the first user to a plurality of teeth of the first user. A second user positions a plurality of sides of the pad within the mouth wherein resting a first side of the plurality of sides on a floor of the mouth and abutting a second side of the plurality of sides against the tongue wherein facing a third side of the plurality of sides towards the plurality of teeth.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a dental cheek and tongue retracting device and method according to an embodiment of the disclosure.

FIG. 2 is a front view of an embodiment of the disclosure.

FIG. 3 is a side view of an embodiment of the disclosure.

FIG. 4 is an in-use view of an embodiment of the disclosure.

FIG. 5 is an alternative in-use view of an embodiment of the disclosure.

FIG. 6 is an in-use view of an embodiment of the disclosure.

### DETAILED DESCRIPTION OF THE INVENTION

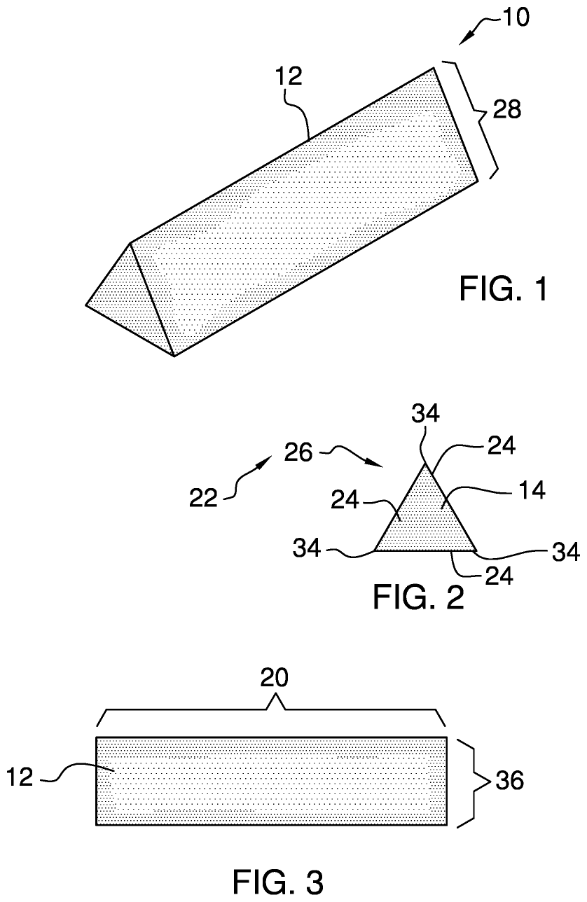

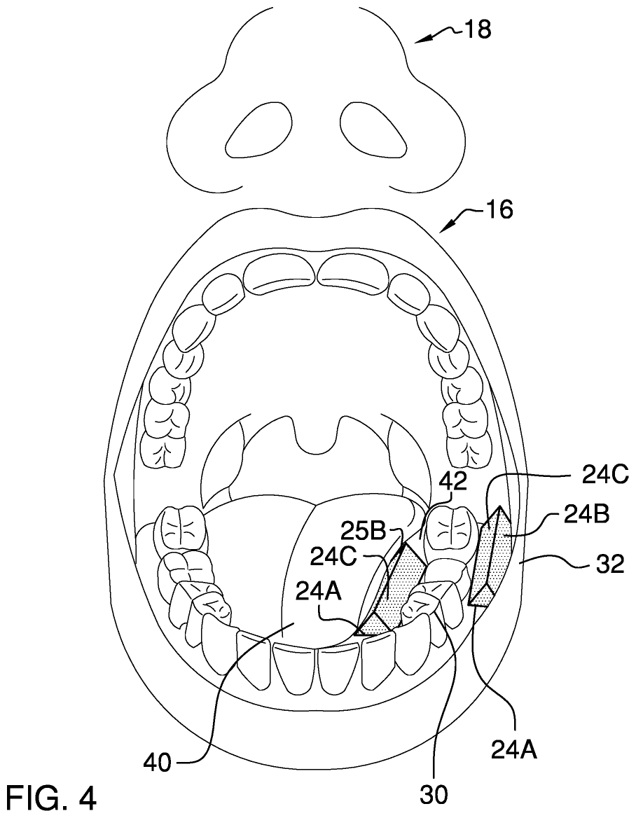

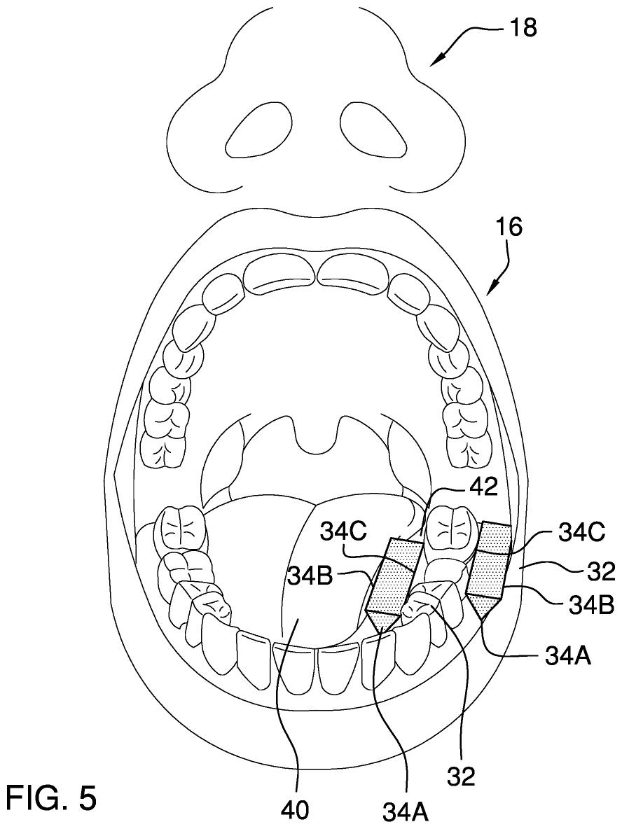

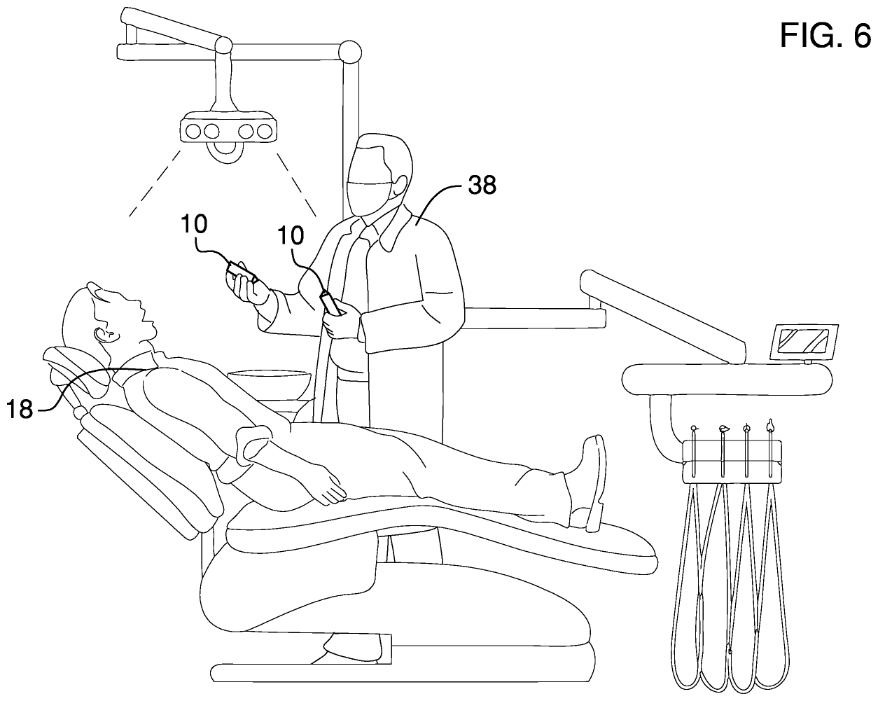

With reference now to the drawings, and in particular to FIGS. 1 through 6 thereof, a new dental retractor device embodying the principles and concepts of an embodiment of the disclosure and generally designated by the reference numeral 10 will be described.

As best illustrated in FIGS. 1 through 6, the dental cheek and tongue retracting device and method 10 generally comprises a pad 12. The pad 12 is configured for being used in a dental procedure such as a crown cementation procedure. The pad 12 is made of a cotton material 14 configured for being absorbent. In addition, the pad 12 is configured for being soft within a mouth 16 of a user 18 wherein providing comfort. The pad 12 has a length 20 being configured for being a range of sizes relative to the mouth 16 of the first user 18. Moreover, the length 20 of the pad 12 is configured for being from twenty-five millimeters to fifty millimeters. Furthermore, the length 20 of the pad 12 is configured for being thirty-eight millimeters.

A triangular shape 22 of the pad 12 includes a plurality of sides 24. Each of the sides 24 is configured for being a flat surface wherein the flat surface of each of the sides 24 is configured for ameliorating in retaining the pad 12 within a fixed position within the mouth 16 of the first user 18. The triangular shape 22 of the pad 12 is an equilateral triangle 26 thus the equilateral triangle 26 is configured for each of the sides 24 of the pad 12 having a width 28. The width 28 of each of the sides 24 is configured for being a range of lengths relative from a plurality of teeth 30 of the first user 18 to the cheek 32 of the first user 18. Moreover, the width 28 of each of the sides 24 is configured for being from eleven millimeters to twelve millimeters. Furthermore, the width 28 of each of the sides 24 is configured for being eleven and one thousand, one hundred twenty-five ten-thousandths millimeters.

A plurality of points 34 of the pad 12 is a junction between the plurality of sides 24 of the triangular shape 22 of the pad 12. Each of the points 34 is positioned parallel relative to a respective one of each of the sides 24 of the triangular shape 22 of the pad 12, thus defining a height 36 of the pad 12. The height 36 of the pad 12 is configured for being a range of heights relative to the plurality of teeth 30 of the first user 18. Moreover, the height 36 of the pad 12 is configured for being from nine millimeters to twelve millimeters. Furthermore, the height 36 of the pad 12 is configured for being nine and five hundred twenty-five thousandths millimeters.

In use, a second user 38 inserts the pad 12 within the mouth 16 of the first user 18 prior to nesting the pad 12 from a tongue 40 of the first user 18 to the plurality of teeth 30 of the first user 18. The second user 38 positions the plurality of sides 24 of the pad 12 within the mouth 16 wherein resting a first side 24A of the plurality of sides 24 on a floor 42 of the mouth 16 and abutting a second side 24B of the plurality of sides 24 against the tongue 40, thus facing a third side 24C of the plurality of sides 24 towards the plurality of teeth 30. Furthermore, the second user 38 can position the plurality of points 34 of the pad 12 within the mouth 16 wherein resting a first point 34A of the plurality of points 34 on the floor 42 of the mouth 16 and abutting a second point 34B of the plurality of points 34 against the tongue 40, thus facing a third point 34C of the plurality of points 34C towards the plurality of teeth 30. Furthermore, the second user 38 can insert the pad 12 within the mouth 16 prior to nesting the pad 12 from the plurality of teeth 30 to the cheek 32 of the first user 18. The second user 38 positions the plurality of sides 24 of the pad 12 within the mouth 16 wherein resting the first side 24A of the plurality of sides 24 on the floor 42 of the mouth 16 and abutting the second side 24B of the plurality of sides 24 against the cheek 32, thus facing the third side 24C of the plurality of sides 24 towards the plurality of teeth 30. Additionally, the second user 38 can position the plurality of points 34 of the pad 12 within the mouth 16 wherein resting the first point 34A of the plurality of points 34 on the floor 42 of the mouth 16 and abutting the second point 34B of the plurality of points 34B against the cheek 32, thus facing the third point 34C of the plurality of points 34 towards the plurality of teeth 30.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A dental cheek and tongue retracting device configured for retracting a tongue and a cheek from a plurality of teeth of the first user, the dental cheek and tongue retracting device comprising:
a pad being configured for being used in a dental procedure, the pad being made of a cotton material, the pad being configured for being soft within a mouth of a user wherein providing comfort;
a triangular shape of the pad, the triangular shape of the pad including a plurality of sides, each of the sides being configured for being a flat surface, the flat surface of each of the sides being configured for ameliorating in retaining the pad within a fixed position within the mouth of the first user; and
a plurality of points of the pad, the plurality of points being a junction between the plurality of sides of the triangular shape of the pad.

2. The dental cheek and tongue retracting device of claim 1, further comprising the cotton material configured for being absorbent.

3. The dental cheek and tongue retracting device of claim 1, further comprising the pad having a length.

4. The dental cheek and tongue retracting device of claim 3, further comprising the length being configured for being a range of sizes relative to the mouth of the first user.

5. The dental cheek and tongue retracting device of claim 4, further comprising the length of the pad being configured for being from 25 millimeters to 50 millimeters.

6. The dental cheek and tongue retracting device of claim 5, further comprising the length of the pad being configured for being 38 millimeters.

7. The dental cheek and tongue retracting device of claim 1, further comprising the triangular shape of the pad being an equilateral triangle, the equilateral triangle being configured for each of the sides of the pad having a width.

8. The dental cheek and tongue retracting device of claim 7, further comprising the width of each of the sides being configured for being a range of lengths relative from the plurality of teeth of the first user to the cheek of the first user.

9. The dental cheek and tongue retracting device of claim 8, further comprising the width of each of the sides being configured for being from 11 millimeters to 12 millimeters.

10. The dental cheek and tongue retracting device of claim 9, further comprising the width of the first side being configured for being 11.1125 millimeters.

11. The dental cheek and tongue retracting device of claim 1, further comprising each of the points being positioned parallel relative to a respective one of each of the sides of the triangular shape of the pad wherein defining a height of the pad.

12. The dental cheek and tongue retracting device of claim 11, further comprising the height of the pad being configured for being a range of heights relative to the plurality of teeth of the first user.

13. The dental cheek and tongue retracting device of claim 12, further comprising the height of the pad being configured for being from 9 millimeters to 12 millimeters.

14. The dental cheek and tongue retracting device of claim 13, further comprising the height of the pad being configured for being 9.525 millimeters.

15. A dental cheek and tongue retracting device configured for retracting a tongue and a cheek from a plurality of teeth of the first user, the dental cheek and tongue retracting device comprising:
a pad being configured for being used in a dental procedure, the pad being made of a cotton material, the cotton material configured for being absorbent, the pad being configured for being soft within a mouth of a user wherein providing comfort, the pad having a length, the length being configured for being a range of sizes relative to the mouth of the first user, the length of the pad being configured for being from 25 millimeters to 50 millimeters, the length of the pad being configured for being 38 millimeters;
a triangular shape of the pad, the triangular shape of the pad including a plurality of sides, each of the sides being configured for being a flat surface, the flat surface of each of the sides being configured for ameliorating in retaining the pad within a fixed position within the mouth of the first user, the triangular shape of the pad being an equilateral triangle, the equilateral triangle being configured for each of the sides of the pad having a width, the width of each of the sides being configured for being a range of lengths relative from the plurality of teeth of the first user to the cheek of the first user, the width of each of the sides being configured for being from 11 millimeters to 12 millimeters, the width of each of the sides being configured for being 11.1125 millimeters; and
a plurality of points of the pad, the plurality of points being a junction between the plurality of sides of the triangular shape of the pad, each of the points being positioned parallel relative to a respective one of each of the sides of the triangular shape of the pad wherein defining a height of the pad, the height of the pad being configured for being a range of heights relative to the plurality of teeth of the first user, the height of the pad being configured for being from 9 millimeters to 12 millimeters, the height of the pad being configured for being 9.525 millimeters.

16. A method for retaining a dental cheek and tongue retracting device in a fixed position within a mouth of a user, steps of the method comprising:
inserting a pad within the mouth;
nesting the pad from a tongue of the first user to a plurality of teeth of the first user;
positioning a plurality of sides of the pad within the mouth wherein resting a first side of the plurality of sides on a floor of the mouth;
abutting a second side of the plurality of sides against the tongue; and
facing a third side of the plurality of sides towards the plurality of teeth.
